(19) United States
(12) Patent Application Publication
(10) Pub. No.: US 2021/0059573 A1
(43) Pub. Date: Mar. 4, 2021

(54) ADAPTIVE VITAL-SIGN DETECTION METHOD AND SYSTEM

(71) Applicant: Wistron Corporation, New Taipei City (TW)

(72) Inventor: Hsi-Wen Wang, New Taipei City (TW)

(21) Appl. No.: 16/680,174

(22) Filed: Nov. 11, 2019

(30) Foreign Application Priority Data
Sep. 3, 2019 (TW) 108131755

(51) Int. Cl. A61B 5/11; A61B 5/00; A61B 5/024; A61B 5/08

(52) U.S. Cl. CPC A61B 5/1126; A61B 5/1123; A61B 5/0816; A61B 5/024; A61B 5/7225

(57) ABSTRACT

An adaptive vital-sign detection method includes (a) receiving statuses in a first period, the status being stationary, motion or leave; (b) detecting whether the first period is interfered according to a status percentage in the first period; (c) receiving statuses in a second period if the first period is detected as being interfered, the second period being different from the first period; (d) determining an optimized status as being stationary if the first period is detected as being not interfered; (e) determining the optimized status as being motion or leave according to dynamic change of the statuses in the second period; (f) receiving vital signs in a third period when the optimized status is determined as being stationary or motion; and (g) processing the vital signs in the third period to obtain corresponding vital signs of the optimized status.

### CROSS-REFERENCE TO RELATED APPLICATIONS

This application claims priority of Taiwan Patent Application No. 108131755, filed on Sep. 3, 2019, the entire contents of which are herein expressly incorporated by reference.

### BACKGROUND OF THE INVENTION

### 1. Field of the Invention

The present invention generally relates to a vital-sign detection method, and more particularly to an adaptive vital-sign detection method adaptable to a contact or non-contact detection device.

### 2. Description of Related Art

Body temperature (BT), blood pressure (BP), heart rate (HR) and respiratory rate (RR) are four primary vital signs. The detection of the vital signs may be used to evaluate health condition or provide a clue to illness of a person.

Conventional health detection devices may be divided into two categories: contact and non-contact. The contact detection device, such as Xiaomi Mi band, may be worn on the body and may collect vital signs (e.g., heart rate) via sensors. The non-contact detection device, such as sensing radar, may obtain vital signs (e.g., hear rate or respiratory rate) by transmitting radio-frequency (RF) signals and analyzing reflected RF signals.

The wearable (contact) detection devices may generally have limited computation capability, and thus cannot further process the collected vital signs. The non-contact detection devices, although having more powerful computation capability, may be liable to interference from environmental noise, therefore resulting in misjudgment or frequent switching of status.

A need has thus arisen to propose a novel scheme to overcome drawbacks of conventional contact or non-contact health detection devices.

### SUMMARY OF THE INVENTION

In view of the foregoing, it is an object of the embodiment of the present invention to provide an adaptive vital-sign detection method capable of obtaining more accurate and stable vital signs adaptable to a contact or non-contact detection device.

According to one embodiment, an adaptive vital-sign detection method includes: (a) receiving statuses in a first period, the status being stationary, motion or leave; (b) detecting whether the first period is interfered according to a status percentage in the first period; (c) receiving statuses in a second period if the first period is detected as being interfered, the second period being different from the first period; (d) determining an optimized status as being stationary if the first period is detected as being not interfered; (e) determining the optimized status as being motion or leave according to dynamic change of the statuses in the second period; (f) receiving vital signs in a third period when the optimized status is determined as being stationary or motion; and (g) processing the vital signs in the third period to obtain a corresponding vital sign of the optimized status.

### BRIEF DESCRIPTION OF THE DRAWINGS

FIG. 1 shows a block diagram illustrating an adaptive vital-sign detection system according to one embodiment of the present invention;

FIG. 2 shows a flow diagram illustrating an adaptive vital-sign detection method executable by the second-stage detector of FIG. 1;

FIG. 3 shows plural statuses in the second period;

FIG. 4 exemplifies polarization signal, detected status from the first-stage detector and detected status from the second-stage detector;

FIG. 5 exemplifies polarization signal, detected status from the first-stage detector and detected status from the second-stage detector; and

FIG. 6 lists some cases of determining the optimized status according to the status percentages by using the sliding window.

### DETAILED DESCRIPTION OF THE INVENTION

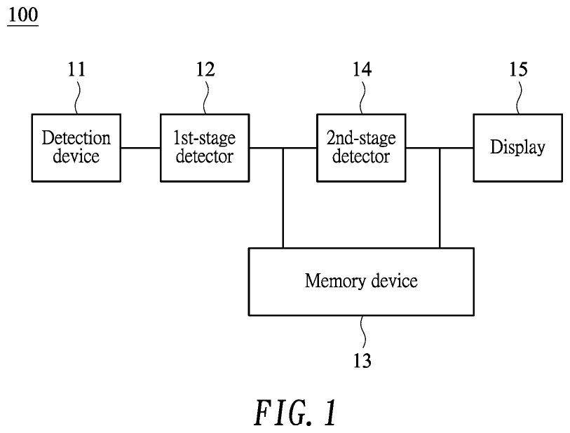

FIG. 1 shows a block diagram illustrating an adaptive vital-sign detection system 100 capable of detecting a vital sign such as heart rate (HR) or respiratory rate (RR) according to one embodiment of the present invention.

In the embodiment, the adaptive vital-sign detection system (detection system hereinafter) 100 may include a (non-contact or contact) detection device 11. In one embodiment, the (non-contact) detection device 11 may include a radar configured to transmit a radio-frequency (RF) signal to a person under detection, followed by receiving a reflected RF signal, which may then be converted to obtain an in-phase polarization signal I and a quadrature polarization signal Q. The radar of the embodiment may be a continuous-wave (CW) radar or an ultra-wideband (UWB) radar (e.g., frequency modulated continuous waveform (FMCW) radar). In another embodiment, the (contact) detection device 11 may include a wearable detection device (e.g., smart bracelet/watch, wrist or arm blood pressure meter or smart cloth/pants), an electrocardiograph electrode patch, an inductive floor mat, a touch sensor, a finger vital-sign sensor, etc. The detection device 11 may include a sensor configured to obtain a vital-sign related signal. Although the detection device 11 is exemplified by a non-contact radar in the following embodiment, it is appreciated that a contact detection device 11 may be used instead.

The detection system 100 of the embodiment may include a first-stage detector 12 coupled to receive an output signal (e.g., the in-phase polarization signal I and the quadrature polarization signal Q) of the detection device 11, according to which a status and a vital sign (e.g., heart rate and respiratory rate) of the person under detection may be outputted. In the embodiment, the status may be one of the following: stationary (e.g., sleep or rest), motion or leave. In one example, stationary, motion and leave may correspond to status values 4, 2 and 0 respectively. In one embodiment, each vital sign outputted from the first-stage detector 12 may have an index representing signal stability of the corresponding vital sign.

The detection system 100 of the embodiment may include a memory device 13, such as static random-access memory (SRAM) or dynamic random-access memory (DRAM), configured to store the status and the vital sign from the first-stage detector 12.

The detection system 100 of the embodiment may include a second-stage detector 14 coupled to receive and optimize the status from the first-stage detector 12, and obtain a corresponding vital sign (e.g., heart rate and respiratory rage) according to the optimized status. The optimized status and the vital sign from the second-stage detector 14 may be stored in the memory device 13.

The detection system 100 of the embodiment may include a display 15 configured to display the optimized status and the vital sign of the second-stage detector 14, or display the status and the vital sign stored in the memory device 13.

In the embodiment, the first-stage detector 12 and the second-stage detector 14 may be two distinct processing devices. Alternatively, in another embodiment, the first-stage detector 12 and the second-stage detector 14 may be integrated into a single processing device. The processing devices mentioned above may be general processors, micro-control units (MCUs), digital signal processors (DSPs) and/or neural processing units (NPUs), which may include a variety of logic circuits configured to provide data processing or computation functions, to store data into or read data from the memory device 13, and to transfer frame data to the display 15.

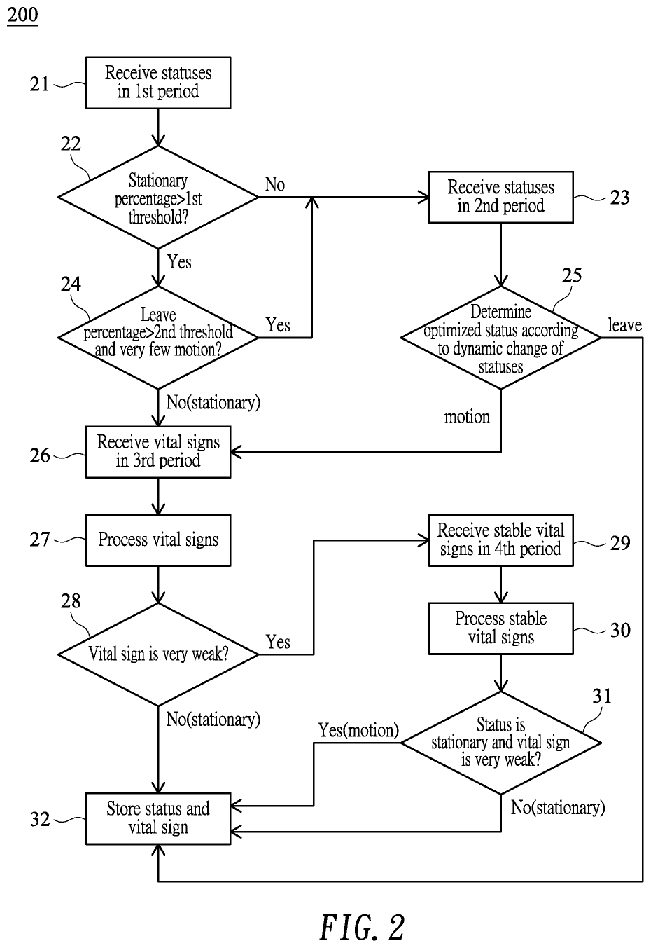

FIG. 2 shows a flow diagram illustrating an adaptive vital-sign detection method (detection method hereinafter) 200 executable by the second-stage detector 14 of FIG. 1. In step 21, statuses in a predetermined first period (e.g., 30 seconds) are received. Next, according to one aspect of the embodiment, steps 22 and 24 are performed to detect whether the first period is environmentally interfered according to a status percentage in the first period, details of which are described as follows.

In step 22, it is determined whether a percentage of stationary status in the first period is greater than a predetermined first threshold (e.g., 60%), which may be set according to specific applications. For example, the first threshold may be set less when environmental interference becomes greater. If a result of step 22 is negative (indicating that a majority of statuses in the first period are motion and leave statuses, which may probably be caused by environmental interference), the flow goes to step 23, in which statuses in a predetermined second period are received, where the second period is different from the first period. In one embodiment, the predetermined second period (e.g., 60 seconds) is greater than the predetermined first period (e.g., 30 seconds).

If the result of step 22 is positive, the flow goes to step 24, in which it is determined whether a percentage of leave status is greater than a predetermined second threshold (e.g., 25%) and there are very few motion statuses (that is, the percentage of motion status is zero, approximately zero or less than a predetermined threshold). It a result of step 24 is positive (indicating that the person under detection may leave without accompanying motion, which may probably be caused by environmental interference), the flow goes to step 23, in which statuses in the predetermined second period are received. If the result of step 24 is negative, an optimized status is determined as being stationary. It is noted that a sequence of performing step 22 and step 24 may be reversed.

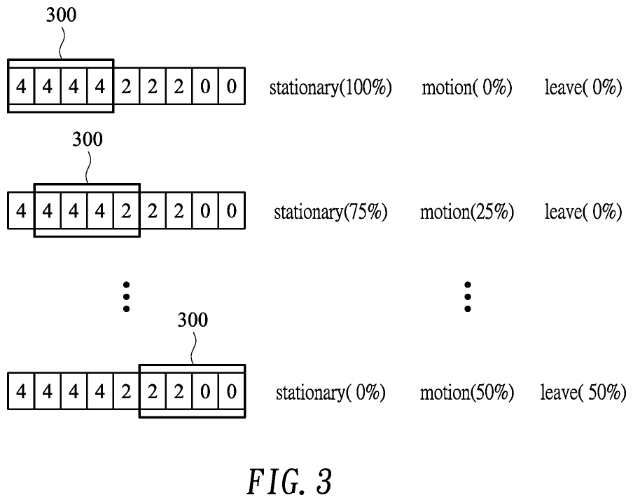

After receiving the statuses in the second period (step 23), the flow goes to step 25 to determine the optimized status. Specifically, according to another aspect of the embodiment, step 25 may determine the optimized status as being motion or leave according to dynamic change of the statuses (over time) in the second period. FIG. 3 shows plural statuses in the second period, where status values 4, 2 and 0 correspondingly represent stationary, motion and leave. As shown in FIG. 3, a sliding window 300 with a predetermined size (e.g., 4) may, in a time sequence, select a group of statuses, according to which a status percentage of each status in said group of statuses may be determined. Subsequently, the sliding window 300 may move to next time to select another group of statuses and determine a status percentage of each status in said another group of statuses. The operation is repetitively performed (predetermined) plural times. In one embodiment, the sliding window 300 may have a size equal to half of the number of statuses in the second period. Generally speaking, the smaller the sliding window 300 is, the more accurate (but slower) the result is; or the larger the sliding window 300 is, the faster (but less accurate) the result is.

In the example shown in FIG. 3, the status percentage of stationary gradually decreases, the status percentage of motion gradually increases, and the status percentage of leave gradually increases, representing a scenario in which a person under detection sleeps or rests (i.e., stationary status) at the beginning, followed by getting up (i.e., motion status), and finally leaving (i.e., leave status) the detected area. If dynamic change of the statuses conforms to this trend, the optimized status is determined as being leave, and the flow then goes to step 32 to store the (optimized) status and corresponding vital sign, for example, in the memory device 13; otherwise, the optimized status is determined as being motion.

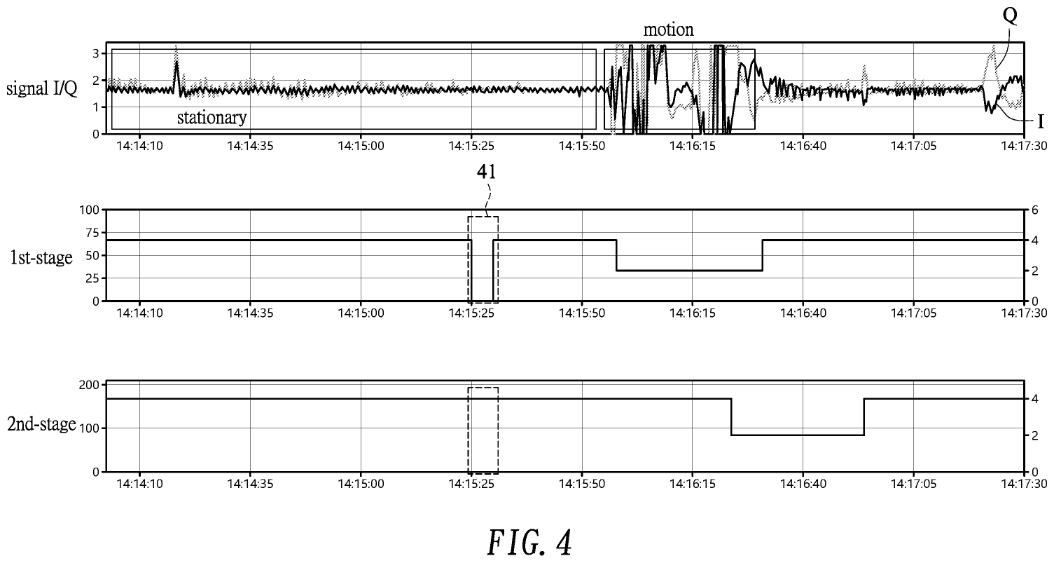

According to the aspect of the embodiment as described above, statuses may be adaptively received in different period (e.g., the first period or the second period) according to the status percentage (step 22 and step 24). Accordingly, status misjudgment due to environmental interference may be prevented. FIG. 4 exemplifies polarization signal I/Q, detected status from the first-stage detector 12 and detected status from the second-stage detector 14. In this example, the first-stage detector 12 generates misjudgment 41 that misjudges stationary status as leave status. However, the second-stage detector 14 may prevent this misjudgment 41.

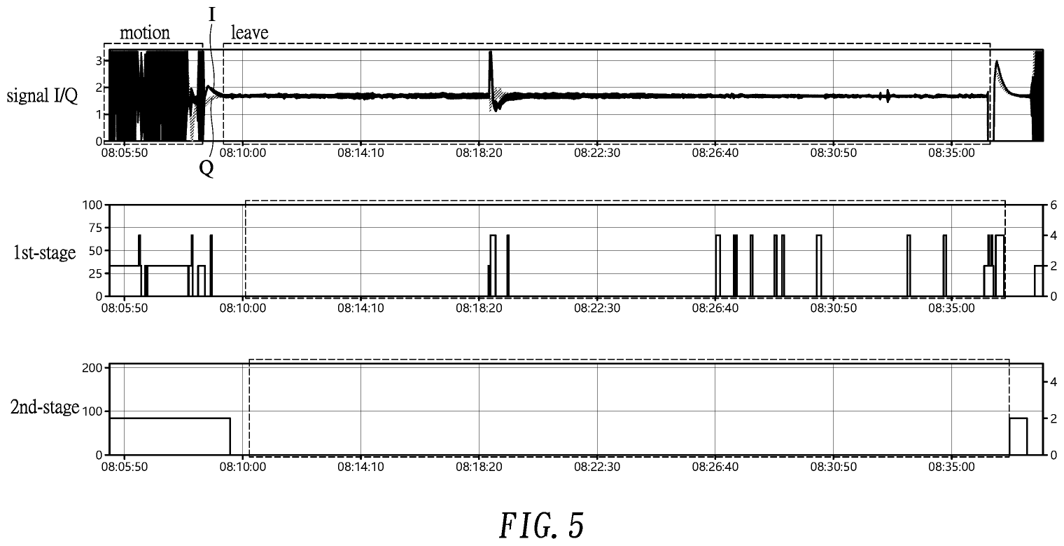

According to another aspect of the embodiment as described above, the optimized status may be correctly determined as being leave by using the sliding window 300 (step 25). FIG. 5 exemplifies polarization signal I/Q, detected status from the first-stage detector 12 and detected status from the second-stage detector 14. In this example, the first-stage detector 12 misjudges leave status as being stationary several times due to environmental noise. However, the second-stage detector 14 may prevent the misjudgments by using the sliding window 300, therefore obtaining the stable status.

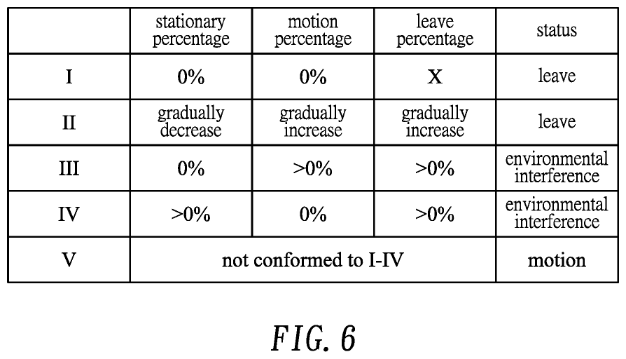

FIG. 6 lists some cases of determining the optimized status according to the status percentages by using the sliding window 300. In case I, as the status percentage of stationary is very small (near or equal to 0%) and the status percentage of motion is very small (near or equal to 0%), the optimized status is thus determined as being leave. In case II, as the status percentage of stationary gradually decreases, the status percentage of motion gradually increases and the status percentage of leave gradually increases, the optimized status is thus determined as being leave. In case III, as the status percentage of stationary is very small (near or equal to 0%), the status percentage of motion is greater than 0% and the status percentage of leave is greater than 0%, this case is disregarded as being environmentally interfered. In case IV, as the status percentage of stationary is greater than 0%, the status percentage of motion is equal to 0% and the status percentage of leave is greater than 0%, this case is disregarded as being environmentally interfered. In case V, as not conformed to cases I-IV, the optimized status is thus determined as being motion.

Referring back to the detection method 200 of FIG. 2, when the optimized status is determined as being stationary (step 24) or motion (step 25), the flow goes to step 26, in which plural vital signs (e.g., heart rate or respiratory rate) may be received in a predetermined third period (e.g., 60 seconds). The first period (step 21), the second period (step 23) and the third period (step 26) may be set according to specific applications. In one embodiment, the detection method 200 may be utilized to monitor the heart rate and the respiratory rate of newborn babies, and the first period may be set to be 30-40 seconds, and the second period and the third period may be set to be 60-100 seconds. In another embodiment, the detection method 200 may be utilized to monitor elderly, and the first period may be set to be 60-90 seconds, and the second period and the third period may be set to be 30-45 seconds.

Next, in step 27, the vital signs (in the third period) may be processed to obtain a vital sign corresponding to the optimized status. In the embodiment, outlier and moving average are adopted to process the vital signs. In one embodiment, outlier may be performed according to average and standard deviation of the vital signs (e.g., heart rate or respiratory rate) received in step 26 as follows. The vital sign Y is deleted if not within the range specified below.

$$\frac{{A}_{1} + {A}_{2} … + {A}_{n}}{n} - X * \sqrt{\frac{{\sum }_{i = 1}^{n} {( {A}_{i} - \overline{A} )}^{2}}{n}} < Y < \frac{{A}_{1} + {A}_{2} … + {A}_{n}}{n} + X * \sqrt{\frac{{\sum }_{i = 1}^{n} {( {A}_{i} - \overline{A} )}^{2}}{n}}$$

where A represents the vital sign, Ā represents an average of the vital signs, and X is a predetermined tolerance value (e.g., 0.5-1). According to the formula shown above, the greater the tolerance value X is, the less outliers are deleted; or the smaller the tolerance value X is, the more outliers are deleted.

After deleting the outliers, the embodiment adopts moving average to process the vital signs left as below.

$${F}_{t} = {MA}_{n} = \frac{{\sum }_{i = 1}^{n} {A}_{t - i}}{n} = \frac{{A}_{t - n} + … + {A}_{t - 2} + {A}_{t - 1}}{n}$$

where Fₜ is a predicted value or a result MAₙ of moving average representing a moving average of n groups of vital signs; n represents the times the moving average performs or the number of the vital signs; Aₜ₋ᵢ represents a value of (t-i)-th vital sign.

Next, according a further aspect of the embodiment, in steps 28-31, it may detect whether the vital signs in the third period are not normally obtained due to movement of the person under detection, thereby determining the optimized status as being stationary or motion.

Specifically, in step 28, it determines whether the vital sign is very weak (i.e., equal to zero, near zero or less than a predetermined threshold). If the result of step 28 is positive (indicating that the vital sign in the third period is not normally obtained due to movement of the person under detection), the flow goes to step 29 to receive (plural) stable vital signs in a predetermined fourth period, where the fourth period is different from the third period. In the embodiment, the predetermined fourth period (e.g., 90 seconds) is greater than the predetermined third period (e.g., 60 second). In the embodiment, the stable vital sign refers to a vital sign having a high index (representing signal stability of the corresponding vital sign). Next, in step 30, the stable vital signs in the fourth period may be processed, for example, by using similar techniques as in step 27, details of which are omitted for brevity. If the result of step 28 is negative (indicating that the vital signs in the third period are not affected by movement of the person under detection), the optimized status is determined as being stationary, followed by going to step 32 to store the (optimized) status and corresponding vital sign, for example, in the memory device 13.

After performing step 30, the flow goes to step 31 to determine whether the (optimized) status is stationary and the vital sign is very weak (i.e., equal to zero, near zero or less than a predetermined threshold). If the result of step 31 is positive (indicating that the vital sign in the third period is not normally obtained due to movement of the person under detection), the optimized status is thus determined as being motion; otherwise the optimized status is determined as being stationary. Subsequently, in step 32, the (optimized) status and corresponding vital sign are stored, for example, in the memory device 13.

Although specific embodiments have been illustrated and described, it will be appreciated by those skilled in the art that various modifications may be made without departing from the scope of the present invention, which is intended to be limited solely by the appended claims.

What is claimed is:

1. An adaptive vital-sign detection method, comprising:
(a) receiving statuses in a first period, the status being stationary, motion or leave;
(b) detecting whether the first period is interfered according to a status percentage in the first period;
(c) receiving statuses in a second period if the first period is detected as being interfered, the second period being different from the first period;
(d) determining an optimized status as being stationary if the first period is detected as being not interfered;
(e) determining the optimized status as being motion or leave according to dynamic change of the statuses in the second period;
(f) receiving vital signs in a third period when the optimized status is determined as being stationary or motion; and
(g) processing the vital signs in the third period to obtain a corresponding vital sign of the optimized status.

2. The method of claim 1, wherein the step (b) comprises:
(b1) determining whether a percentage of stationary status in the first period is greater than a first threshold;
(b2) performing the step (c) if a result of the step (b1) is negative, otherwise determining whether a percentage of leave status is greater than a second threshold and there are very few motion statuses; and
(b3) performing the step (c) if a result of the step (b2) is positive, otherwise determining the optimized status as being stationary.

3. The method of claim 1, wherein the step (e) comprises:
(e1) using a sliding window with a predetermined size to select a group of statuses, according to which a status percentage of each status in said group of statuses is determined;
(e2) moving the sliding window to next time to select another group of statuses and determine the status percentage of each status in said another group of statuses; and
(e3) repetitively performing the step (e2) a predetermined number of times;
wherein the optimized status is determined as being leave if the status percentage of stationary gradually decreases, the status percentage of motion gradually increases, and the status percentage of leave gradually increases.

4. The method of claim 1, wherein the step (g) comprises:
deleting outliers of the vital signs in the third period; and
using moving average to process the vital signs left after deleting the outliers.

5. The method of claim 1, further comprising:
storing the optimized status and the corresponding vital sign.

6. The method of claim 1, after the step (g) further comprising:
(h) detecting whether the vital sign in the third period is not normally obtained due to movement of a person under detection, thereby determining the optimized status as being stationary or motion.

7. The method of claim 6, wherein the step (h) comprises:
(h1) determining whether the vital sign is very weak;
(h2) if a result of the step (h1) is positive, receiving stable vital signs in a fourth period, the fourth period being different from the third period, otherwise determining the optimized status as being stationary;
(h3) processing the stable vital signs in the fourth period;
(h4) determining whether the optimized status is stationary and the corresponding vital sign is very weak; and
(h5) if a result of the step (h4) is positive, determining the optimized status as being motion, otherwise determining the optimized status as being stationary.

8. The method of claim 7, wherein the stable vital sign is selected according to a corresponding index representing signal stability of the corresponding vital sign.

9. The method of claim 1, wherein the vital sign comprises a heart rate or a respiratory rate.

10. An adaptive vital-sign detection system, comprising:
a detection device;
a first-stage detector that receives an output signal of the detection device, and accordingly outputs a status and a corresponding vital sign, the status being stationary, motion or leave; and
a second-stage detector that receives and optimizes the status from the first-stage detector to generate an optimized status, according to which a corresponding vital sign is obtained;
wherein the second-stage detector performs the following steps:
(a) receiving statuses in a first period;
(b) detecting whether the first period is interfered according to a status percentage in the first period;
(c) receiving statuses in a second period if the first period is detected as being interfered, the second period being different from the first period;
(d) determining the optimized status as being stationary if the first period is detected as being not interfered;
(e) determining the optimized status as being motion or leave according to dynamic change of the statuses in the second period;
(f) receiving vital signs in a third period when the optimized status is determined as being stationary or motion; and
(g) processing the vital signs in the third period to obtain the corresponding vital sign of the optimized status.

11. The system of claim 10, wherein the detection device comprises a radar that transmits a radio-frequency signal to a person under detection, followed by receiving a reflected radio-frequency signal.

12. The system of claim 10, wherein the step (b) comprises:
(b1) determining whether a percentage of stationary status in the first period is greater than a first threshold;
(b2) performing the step (c) if a result of the step (b1) is negative, otherwise determining whether a percentage of leave status is greater than a second threshold and there are very few motion statuses; and
(b3) performing the step (c) if a result of the step (b2) is positive, otherwise determining the optimized status as being stationary.

13. The system of claim 10, wherein the step (e) comprises:
(e1) using a sliding window with a predetermined size to select a group of statuses, according to which a status percentage of each status in said group of statuses is determined;
(e2) moving the sliding window to next time to select another group of statuses and determine the status percentage of each status in said another group of statuses; and
(e3) repetitively performing the step (e2) a predetermined number of times;
wherein the optimized status is determined as being leave if the status percentage of stationary gradually decreases, the status percentage of motion gradually increases, and the status percentage of leave gradually increases.

14. The system of claim 10, wherein the step (g) comprises:
deleting outliers of the vital signs in the third period; and
using moving average to process the vital signs left after deleting the outliers.

15. The system of claim 10, further comprising:
storing the optimized status and the corresponding vital sign.

16. The system of claim 10, after the step (g) further comprising:
(h) detecting whether the vital sign in the third period is not normally obtained due to movement of a person under detection, thereby determining the optimized status as being stationary or motion.

17. The system of claim 16, wherein the step (h) comprises:
(h1) determining whether the vital sign is very weak;
(h2) if a result of the step (h1) is positive, receiving stable vital signs in a fourth period, the fourth period being different from the third period, otherwise determining the optimized status as being stationary;
(h3) processing the stable vital signs in the fourth period;
(h4) determining whether the optimized status is stationary and the corresponding vital sign is very weak; and
(h5) if a result of the step (h4) is positive, determining the optimized status as being motion, otherwise determining the optimized status as being stationary.

18. The system of claim 17, wherein the stable vital sign is selected according to a corresponding index representing signal stability of the corresponding vital sign.

19. The system of claim 10, wherein the vital sign comprises a heart rate or a respiratory rate.

20. The system of claim 10, wherein the detection device is a non-contact detection device or a contact detection device.
